(19) United States
(12) Patent Application Publication
(10) Pub. No.: US 2011/0264223 A1
(43) Pub. Date: Oct. 27, 2011

(54) SELF-ADJUSTING AND SELF-STABILIZING INTERVERTEBRAL DISC PROTHESIS

(76) Inventors: Jean-Philippe Lemaire, Saulon la Chapelle (FR); Francois Lavaste, Saint-Michel-sur-Orge (FR)

(21) Appl. No.: 13/057,983

(22) PCT Filed: Aug. 7, 2009
(86) PCT No.: PCT/FR09/00992

§ 371 Date: Apr. 28, 2011

(30) Foreign Application Priority Data
Aug. 7, 2008 (FR) 0804508

(51) Int. Cl. A61F 2/44

(52) U.S. Cl. 623/17.16

(57) ABSTRACT

The present invention relates to an intervertebral disc prosthesis (1) comprising at least three components, namely a first plate, called the upper plate (2), a second plate, called the lower plate (3), and a movable core (4) arranged between said plates (2, 3), characterized in that the movable core (4) comprises a concave upper surface (44) in congruent contact with the convex surface of the upper plate (2) and a convex lower surface (45) in congruent contact with the concave surface of the lower plate (3), each of said surfaces being defined by a distinct radius of curvature, the radius of curvature of the concave surface (44) of the core being less than that of its convex surface (45), the centres of said concave (44) and convex (45) surfaces being situated on the radial axis of symmetry of the central core and being placed on the same side of the prosthesis (1), namely that of the upper plate (2).

The present invention relates to an intervertebral disc prosthesis to take the place of fibrocartilage discs providing a connection between the vertebrae of the spinal column, and more specifically on the cervical spine.

In this field, a large number of prior prostheses have already been produced. However, some of these prostheses may cause adverse effects in patients fitted with said prostheses. Indeed, if they are made of an excessively compressible material or if they allow the various constituent components of said prostheses to move excessively in relation to each other, there is a risk of ejection of at least a portion of the prosthesis from the vertebrae. Furthermore, the literature shows that the majority of these prostheses do not reproduce the kinematics of the vertebral unit correctly with progressive segmental kyphosis positioning explaining the load stress applied to the posterior interapophyseal joints (commonly referred to as posterior joints) giving rise to a high risk of the development of arthrosis in these posterior joints. The main difficulty is that of succeeding in controlling the relative movements of the various constituent components of these prostheses.

It should be noted that the Functional Spinal Unit (FSU) is a complex system including the intervertebral disc, the posterior joints and the musculoligamentous system. This complex system does not have a horizontal transverse plane of symmetry: indeed, in anatomical terms, in the sagittal plane, the contour of the overlying vertebra has a concave trough shape whereas the contour of the upper surface of the underlying vertebra is plane overall. The cavity in the lower surface of the overlying vertebra is pronounced at the cervical vertebrae, whereas it is less pronounced at the lumbar vertebrae. The intervertebral disc between these two surfaces thus does not have a horizontal transverse symmetry.

Since this unit is integrated into an assembly, the sagittal equilibrium minimises the stress on the Posterior Ligamentous System, and thus on the functional unit, helping define the Neutral Zone (mobility zone not involving intrinsic mechanical resistance of the various components).

Degenerative disease is the result of a loss of discal physiological rigidity. Mobility is resolved in an elementary movement, particularly in translation converting the guide function of the joints into an abutment function.

Any intervertebral prosthesis will thus involve a first constraint not only of restoring a mobility amplitude but also preserving or restoring the neutral zone, i.e. an amplitude/physiological neutral zone ratio. This ratio involves the concept of ICR (instant centre of rotation) cohesion with those of the FSU.

The second constraint is that of controlling couplings, i.e. controlling the joint load, particularly if a pathological joint is involved with an impaired posterior ligamentous system, giving rise to modifications in the sagittal equilibrium, and helping extend the Neutral Zone. Therefore, it is necessary to impede flexion-extension postero-anterior translation, by reducing the translation-rotation ratio, and inducing self-centring by means of antero-posterior translation. The same principle applies for the lateral inclination with opposite translation induced by rotation.

In the prior art, intervertebral prostheses are known, comprising three components, namely a first plate, called the upper plate, a second plate, called the lower plate, and a movable core arranged between said plates. The movable core of these prostheses comprises a convex upper contact surface with the upper plate and a concave lower contact surface with the lower plate. Examples of such prostheses can be found in the documents WO 2005/094737, WO 2006/105603, US 2007/270958 or US 2004/138753.

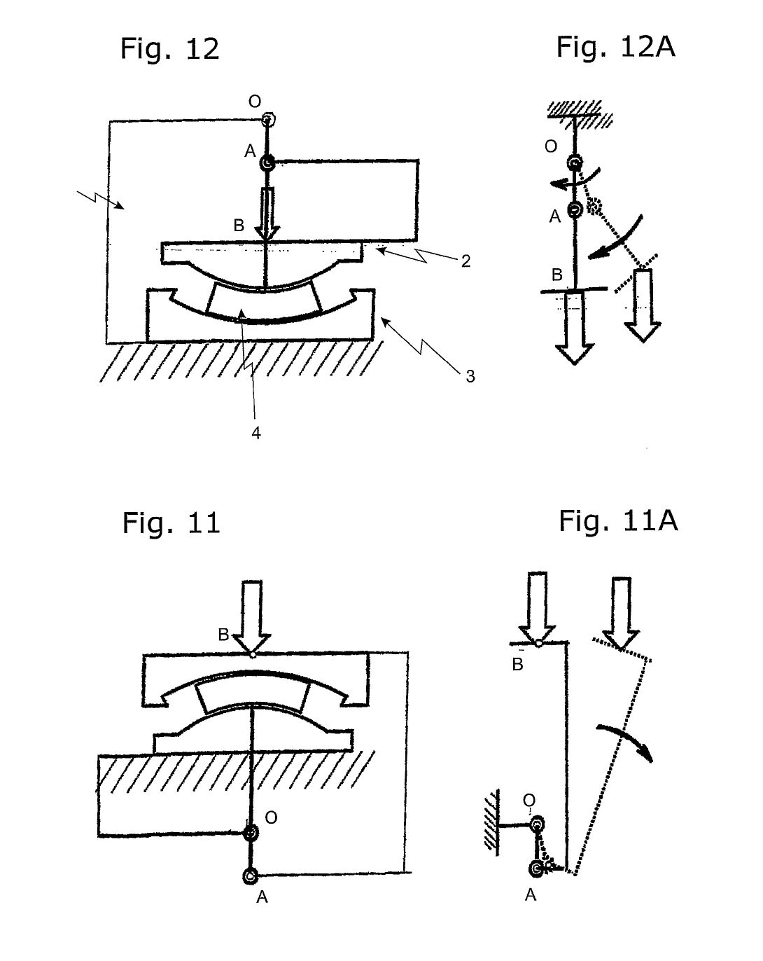

However, these known prostheses do not meet the constraints mentioned above in a satisfactory manner, particularly in that they are not stable in physiological compression scenarios. This failing will be explained in detail further on in the description, with reference to FIGS. 10, 10A, 11 and 11A.

The aim of the present invention is thus that of remedying some drawbacks of the prior art by proposing a novel type of intervertebral disc with a simple design, enabling control of the movements of the various constituent components of said prosthesis in order to avoid subjecting the posterior joints to excessive load stress. Furthermore, the prosthesis according to the invention has kinematics self-centring and self-stabilising the prosthetic components, ensuring a physiological location of the instant centres of rotation.

In this respect, the present invention relates to an intervertebral disc prosthesis comprising at least three components, namely a first plate, called the upper plate, comprising an upper face having a convex profile in the sagittal plane of the prosthesis, a second plate, called the lower plate, comprising a lower face having a plane profile in the sagittal plane of the prosthesis, and a movable core arranged between said plates.

This prosthesis is characterised in that the movable core comprises a concave upper surface in congruent contact with the convex surface provided in the lower surface of the upper plate and a convex lower surface in congruent contact with the concave surface provided in the upper surface of the lower plate, each of said surfaces being defined by a distinct radius of curvature, the radius of curvature of the concave surface of the core being less than that of the convex surface thereof, the centres of said concave and convex surfaces being situated on the radial axis of symmetry of the central core and being placed on the same side of the prosthesis, namely that of the upper plate.

The disc prosthesis according to the present invention has three components with concave and convex surfaces. To meet the physiological constraints mentioned above, the arrangement of these concave and convex surfaces is specific and the inversion thereof cannot be envisaged, due to the considerable advantages offered by this specific configuration, as detailed hereinafter.

A preferential embodiment of the prosthesis according to the invention will be described hereinafter, as a non-limitative example, with reference to the appended figures wherein:

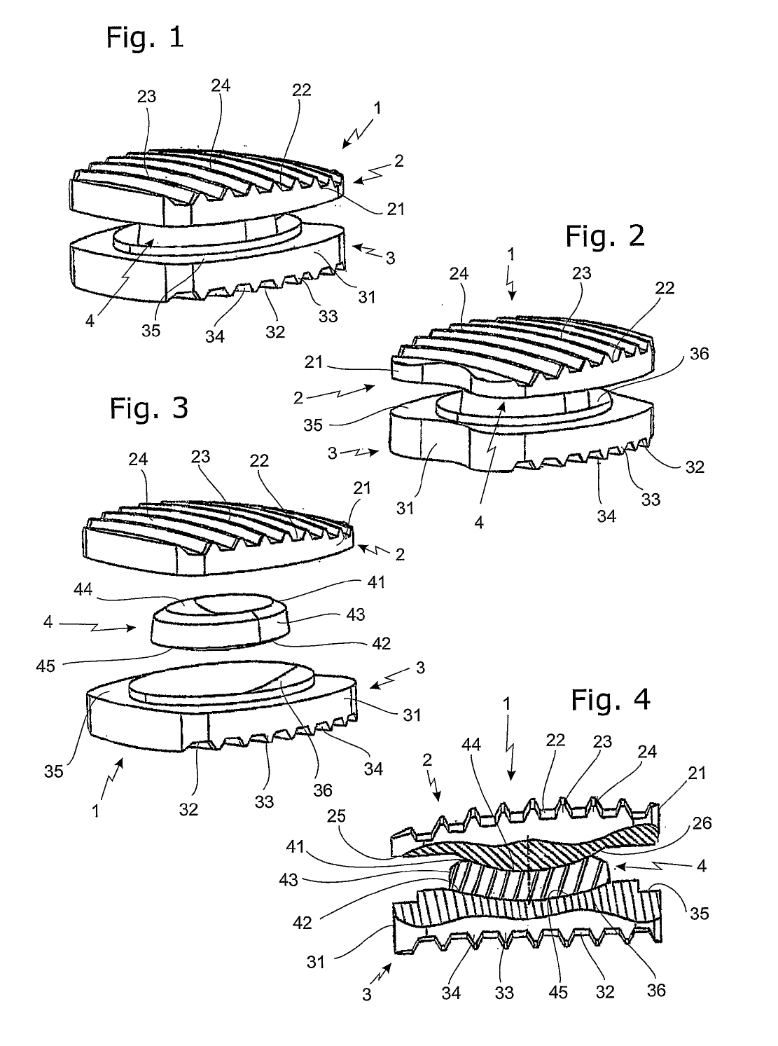

FIG. 1 is a front perspective view of the assembled prosthesis in an upper 3/4 angle,

FIG. 2 is a rear perspective view of the assembled prosthesis in an upper 3/4 angle,

FIG. 3 is an exploded perspective front view in an upper 3/4 angle,

FIG. 4 is a partial sectional view in the sagittal plane of the prosthesis,

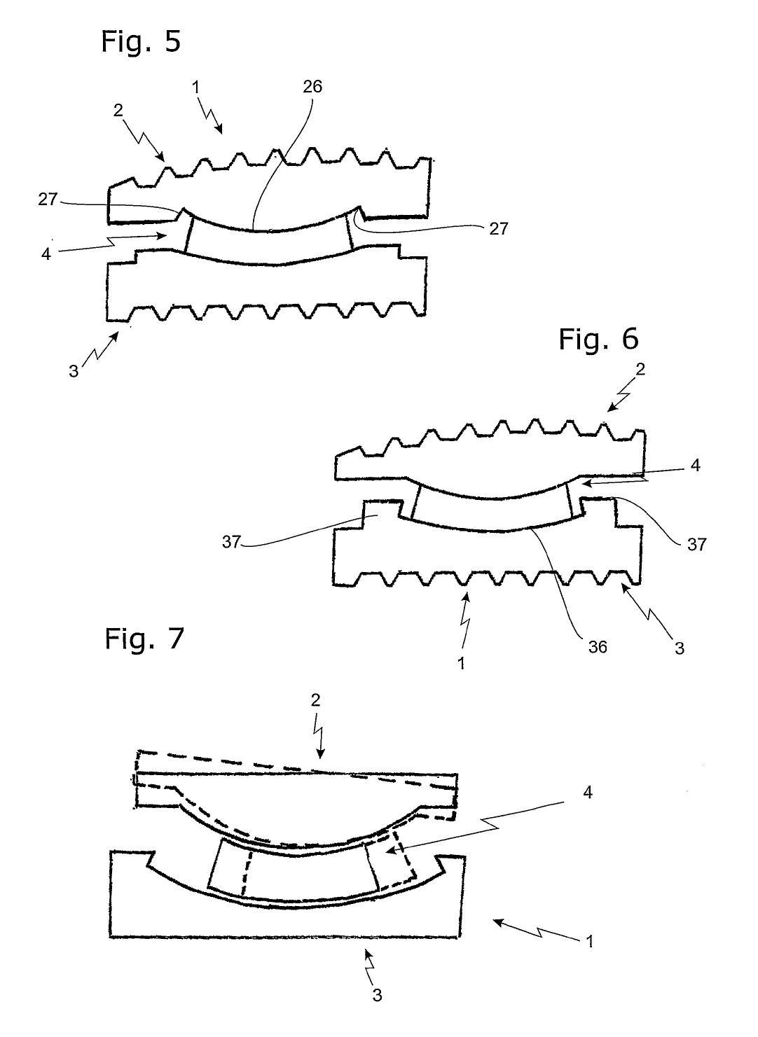

FIG. 5 is a schematic view similar to FIG. 4 representing a first alternative embodiment of the prosthesis with abutments,

FIG. 6 is a schematic view similar to FIG. 4 representing a second alternative embodiment of the prosthesis with abutments,

FIG. 7 is a schematic view similar to FIG. 4 representing compensation of the movements of the various components of the prosthesis in a flexion-extension scenario,

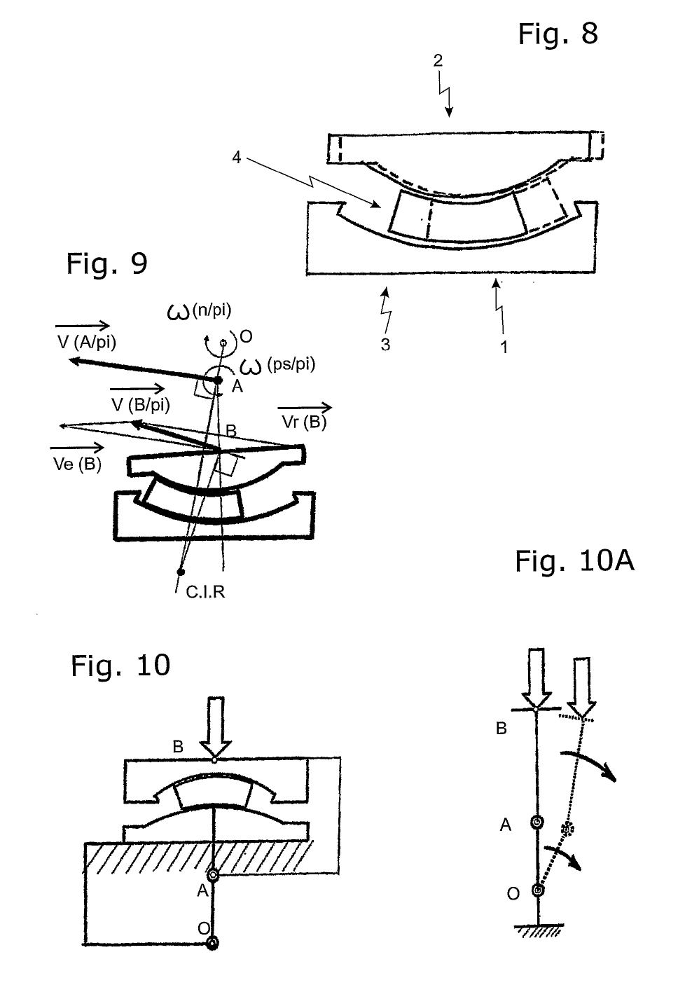

FIG. 8 is a schematic view similar to FIG. 4 representing compensation of the movements of the various components of the prosthesis in a pure translation scenario,

FIG. 9 is a schematic view similar to FIG. 4 representing the location of the instant centre of rotation of the prosthesis in a flexion scenario;

FIGS. 10 and 10A are, respectively, a partial sectional view and a schematic view in the sagittal plane of a prosthesis according to the prior art, where the radius of convexity of the lower plate is greater than the radius of concavity of the upper plate, for FIG. 10A said prosthesis being represented at rest with a solid line and under the effect of compression with a dotted line;

FIGS. 11 and 11A are, respectively, a partial sectional view and a schematic view in the sagittal plane of a prosthesis according to the prior art, where the radius of convexity of the lower plate is less than the radius of concavity of the upper plate, for FIG. 11A said prosthesis being represented at rest with a solid line and under the effect of compression with a dotted line;

FIGS. 12 and 12A are, respectively, a partial sectional view and a schematic view in the sagittal plane of the prosthesis according to the invention, for FIG. 12A said prosthesis being represented at rest with a solid line and under the effect of compression with a dotted line.

With reference to FIGS. 1 to 4, the intervertebral disc prosthesis 1, according to the invention, comprises at least three components, namely a first plate, called the upper plate 2, a second plate, called the lower plate 3, and a movable core 4 arranged between said two plates 2, 3, the upper plate 2 and the lower plate 3 being linked with respect to each other.

In this way, the upper plate 2 comprises an overall flat central body 21, the shape and dimensions of which correspond to those of the lower surface of the vertebra of the cervical spine situated above said upper plate 2. In order to adapt to said vertebra and enable the anchoring of said upper plate 2 on the bone, the upper face 22 of said central body 21 has a slightly rounded convex profile along the sagittal plane. Preferably, the profile along the front plane of this upper face 22 is also convex. The profile in the front plane may also be plane overall: those skilled in the art will adapt the profile of the external upper face 22 of the upper plate 2 to mould the anatomical contour of the associated joint surface, according to the positioning along the spinal column.

Moreover, this upper face 22 is provided with anchoring means 23. In one preferential embodiment, the anchoring means 23 are teeth 24 protruding perpendicularly from said upper face 22, substantially parallel with each other, perpendicular to the sagittal plane of the prosthesis 1. The cross-section of said teeth 24 has a general regular trapezium shape. The upper plate 2 also comprises on at least one portion of the lower face 25 of said central body 21 a convex surface 26, advantageously in the form of a spherical cap arranged in the vicinity of the middle of said lower face 25.

Similarly, the lower plate 3 comprises an overall flat central body 31, the shape and dimensions of which correspond to those of the lower surface of the vertebra of the cervical spine situated above said lower plate 3. In order to adapt to said vertebra and enable the anchoring of said lower plate 3 on the bone, the lower face 32 of said central body 31 has a plane sagittal profile. Preferably, the profile along of the lower face 32 in the front plane is also plane. The profile in the front plane may also be convex, with slight rounding: those skilled in the art will adapt the profile of the external lower face 32 of the lower plate 3 to mould the anatomical contour of the associated joint surface, according to the positioning along the spinal column.

Moreover, this external lower face 32 is provided with anchoring means 33. In one preferential embodiment, the anchoring means 33 are teeth 34 similar to the teeth 24 of the upper plate 2 described above, fitted perpendicularly to the sagittal plane of the prosthesis 1. The lower plate 3 also comprises on at least one portion of the upper face 35 of said central body 31 a concave surface 36, advantageously in the form of a spherical cap arranged in the vicinity of the middle of said upper face 35.

To improve the contact and engagement in the bone of the teeth 24, 34, it is possible, for example, to have a hydroxyapatite type interface.

The movable core 4 comprises an upper face 41, a lower face 42 and a peripheral face 43 interconnecting the upper and lower faces 41, 42. In one preferential embodiment, this peripheral face 43 has an overall tapered shape.

To obtain a joint between the upper plate 2 and the lower plate 3 about the movable core 4, it is understood that the upper plate 2, the lower plate 3 and the movable core 4 should be such that the upper face 41 of the movable core 4 is in congruent contact with the convex surface 26 of the lower face 25 of the upper plate 2 and that the lower face 42 of the movable core 4 is in congruent contact with the concave surface 36 of the upper face 35 of the lower plate 3. Such a configuration makes it possible to obtain relative movement between the upper and lower plates 2, 3 and the movable core 4.

For this purpose, the movable core 4 comprises, firstly, on at least one portion of the upper face 41 thereof, a spherical concave surface 44 having a radius of curvature substantially equal to that of the convex surface 26 of the lower surface 25 of the upper plate 2, and, secondly, on at least one portion of the lower face 41 thereof, a spherical convex surface 45 having a radius of curvature substantially equal to that of the concave surface 36 of the upper surface 35 of the lower plate 3.

In one preferential embodiment, the concave surface 44 and the convex surface 45 cover the entire upper face 41 and lower face 42 of the movable core 4, respectively.

Furthermore, the movable core 4 preferably has a radial axis of symmetry so as to have the centres of the radii of curvature of the upper and lower faces 41, 42 thereof situated on said axis of symmetry and placed on the same side of the prosthesis, namely that of the upper plate 2.

Finally, the radius of curvature of the lower face 42 is greater than that of the upper face 41 and the distance between the centres of the radii of curvature, which is determined by the intervertebral space, is advantageously as small as possible.

Those skilled in the art will have no difficulty in sizing said radii of curvature and, consequently, obtaining relative movement velocities of the various constituent components of the prosthesis 1, enabling said prosthesis 1 to be self-adjusting and self-stabilising. Furthermore, it is understood that said radii may be variable according to the position of the prosthesis 1 along the spinal column, since the relative movement velocities are also dependent on said position.

According to a first alternative embodiment and with reference to FIG. 5, to limit the risk of ejection of at least one portion of the prosthesis 1 and the clearance of one plate with respect to the other, the upper plate 2 comprises, at the periphery of the convex surface 26 thereof on the lower face 25 thereof, abutments in at least both the latero-lateral and antero-posterior directions with respect to the positioning of the prosthesis 1 on the spinal column. These abutments are advantageously obtained by means of a groove 27 produced in the lower face 25 about the spherical convex surface 26 thereof.

Similarly, with reference to FIG. 6, according to a second alternative embodiment, the lower plate 3 comprises, at the periphery of the concave surface 36 thereof on the upper face 35 thereof, abutments in at least both the latero-lateral and antero-posterior directions with respect to the positioning of the prosthesis 1 on the spinal column. These abutments are advantageously obtained by means of a peripheral edge 37 produced on the upper face 35 about the spherical concave surface 36 thereof.

The specific configuration of the prosthesis enables self-centring of the movable core 4 and self-adjustment of the prosthesis 1 so as to observe the natural physiological kinematics of the cervical spine.

Indeed, with reference to FIGS. 7 and 8, representing, respectively, the prosthesis 1 subject to flexion-extension and pure translation deformation, during the movement of the cervical spine and under the action of the forces transmitted by the adjacent vertebrae, the prosthesis 1 changes, for example, from a resting position, representing with a bold line in the figure, to a working position, represented with a dotted line in the figure. It is understood that the movable core 4, when moving, compensates for the movement of the upper plate 2 in relation to the lower plate 3. The prosthesis 1, according to the invention, thus enables control and limitation of the forces applied on the anterior and posterior joints, thus preventing hyperpressure problems.

Furthermore, with reference to FIG. 9, the instant centres of rotation (ICR) of the prosthetic assembly inserted in the intervertebral joints are, for this reason, located in the physiological cloud, whether in flexion extension (FIG. 9) or lateral inflexion (not shown).

With reference to FIG. 9, the ICR is determined as follows: A and B are 2 points belonging to the upper plate. The I.C.R. of the upper plate is situated at the intersection of the perpendiculars to the velocities of these two points (in relation to the lower plate). The core is hereinafter referenced with the letter c, the lower plate with the letters lp and the upper plate with the letters up.

The velocity of A/lower plate (V(A/lp)) is perpendicular to OA, the I.C.R is thus situated on OA.

The velocity of B/lower plate consists of the entrainment velocity Ve(B)=V(A/lp)=OA×ω(c/lp) and the relative velocity perpendicular to AB, Vr(B)=AB×ω(up/lp).

This gives V(B/lp)=Ve(B)+Vr(B).

The I.C.R. is at the intersection of OA and the perpendicular in B to V(B/lp).

Although, in this configuration, the centres of rotation of the mechanical components are above the upper plate, there is cohesion between the prosthetic ICRs and those of the Functional Spinal Unit.

FIGS. 10, 10A and 11, 11A demonstrate the overall physiological instability of the prostheses according to the known prior art, disclosed in the documents WO 2005/094737, WO 2006/105603, US2007/270958 or US 2004/138753, whether with a radius of curvature of the convexity of the lower plate greater (FIGS. 11, 11A) or less (FIGS. 10, 10A) than the radius of curvature of the concavity of the upper plate.

The core moves about the centre O of the convexity of the lower plate and the upper plate moves about the centre A of the convexity of the upper surface of the core.

In the case of the arrangement in FIGS. 10, 10A, when the upper plate is loaded in the middle B thereof, the centre A moves about and above the centre O and the point B moves about and above the centre A and there is nothing preventing the continued movement of the point B about the centre A and the centre A about the centre O without being able to return spontaneously to the original position thereof. Only muscle actions can counteract this instability, inducing a detrimental joint overload and energy expenditure.

In the case of the arrangement in FIGS. 11, 11A, when the upper plate is loaded in the middle B thereof, the centre A moves about and above the centre O and the point B moves about and above the centre A and there is nothing preventing the continued movement of the point B about the centre A and the centre A about the centre O without being able to return spontaneously to the original position thereof. Only muscle actions can counteract this instability, inducing a detrimental joint overload and energy expenditure.

This instability of known prostheses is also found in the bi-convex or bi-concave core prosthesis concept.

With reference to FIGS. 12 and 12A, it will now be demonstrated that, unlike known prostheses, the prosthesis according to the invention is dynamically stable.

In the prosthesis according to the invention, the concave-convex surfaces of the movable core are inverted in relation to the known prior art: the upper plate has a convex spherical surface, the lower plate has a concave spherical surface, and the lens-shaped movable core has concave and convex spherical surfaces in the upper and lower portions respectively.

The radius of curvature r of the concave upper surface of the core, congruent with the convex surface of the upper plate, is less than the radius of curvature R of the convex lower surface, in turn congruent with the concave surface of the lower plate. The thickness of the core is “t”.

In this configuration (see FIG. 12A), the core still moves about the centre O of the concavity of the lower plate, the centre O being situated above the core and the centre A of the concavity of the core moves on a spherical surface portion having the centre O and the radius OA=R−(r+t). When the upper plate is loaded in the middle B thereof, the centre A is moved about and below the centre O, the point B moves about and below the centre A, the system does not give way and the centre A and the point B can return spontaneously to the original position thereof, with no joint overload and no energy expenditure.

This concept positioning the centre of rotation O of the core in relation to the lower plate and the centre of rotation A of the upper plate in relation to the core above the upper plate, with A below O, ensures the physiological stability of the prosthetic assembly.

According to a further feature of the invention, the upper and lower plates 2, 3 and the movable core 4 are advantageously made from non-metallic materials to make it possible to conduct MRIs to monitor the spinal cord in particular. In this way, preferably, the upper and lower plates 2, 3 are made of polyetheretherketone and the movable core 4 made of ceramics. These two materials also offer the advantage of having low friction coefficient between them enabling easy sliding of the components against each other and thus a satisfactory joint of the upper and lower plates 2, 3 on the movable core 4.

Those skilled in the art will have no difficulty in sizing the various constituent components of the prosthesis according to the invention, particularly observing the minimum thicknesses associated with the nature of the materials used. In this way, for example, for the lower plate 3, a value of not less than 1.3 millimetres between the bottom of the concave shape 35 and the lower face 32 will be chosen.

Furthermore, it is understood that, according to the patient's morphology and particularly the intervertebral space, it is necessary to have a prosthesis available in various sizes. In order to adjust the total height of the prosthesis 1, the thickness of the movable core 4 and/or the thickness of the upper plate 2 will be modified.

Finally, the present invention is obviously not limited to the example of a preferential embodiment and the implementation described, and may be modified or adapted according to specific needs or requirements, without leaving the scope of the invention.

1. An intervertebral disc prosthesis comprising at least three components, namely a first plate, called the upper plate, comprising an upper face having a convex profile in the sagittal plane of the prosthesis, a second plate, called the lower plate, comprising a lower face having a plane profile in the sagittal plane of the prosthesis, and a movable core arranged between said plates, wherein the movable core comprises a concave upper surface in congruent contact with the convex surface provided in the lower surface of the upper plate and a convex lower surface in congruent contact with the concave surface provided in the upper surface of the lower plate, each of said surfaces being defined by a distinct radius of curvature, the radius of curvature of the concave surface of the core being less than that of the convex surface thereof, the centres of said concave and convex surfaces being situated on the radial axis of symmetry of the central core and being placed on the same side of the prosthesis, namely that of the upper plate.

2. The intervertebral disc prosthesis according to claim 1, wherein the upper plate comprises a central body comprising on the upper face thereof anchoring means and in the vicinity of the middle of the lower face thereof a convex surface in the form of a spherical cap wherein the radius of curvature is substantially equal to that of the concave surface of the movable core.

3. The intervertebral disc prosthesis according to claim 1, wherein the lower plate comprises a central body comprising on the lower face thereof anchoring means and in the vicinity of the middle of the lower face thereof a convex surface in the form of a spherical cap wherein the radius of curvature is substantially equal to that of the concave surface of the movable core.

4. The intervertebral disc prosthesis according to claim 1, wherein the anchoring means are teeth that are parallel with each other, perpendicular to the sagittal plane of said prosthesis.

5. The intervertebral disc prosthesis according to claim 1, wherein the lower plate comprises abutments in at least both the latero-lateral and antero-posterior directions.

6. The intervertebral disc prosthesis according to claim 1, wherein the abutments are obtained by means of a peripheral edge produced on the upper face of said lower plate about the spherical concave surface.

7. The intervertebral disc prosthesis according to claim 1, wherein the upper plate comprises abutments in at least both the latero-lateral and antero-posterior directions.

8. The intervertebral disc prosthesis according to claim 1, wherein the abutments are obtained by means of a groove produced in the lower face about the convex surface thereof.

9. The intervertebral disc prosthesis according to claim 1, wherein the upper and lower plates are made of polyehteretherketone.

10. The intervertebral disc prosthesis according claim 1, wherein the movable core is made of ceramic.
